(19) United States
(12) Patent Application Publication
(10) Pub. No.: US 2026/0345647 A1
(43) Pub. Date: Nov. 5, 2026

(54) AST-3424 COMBINATION FOR TREATING LEUKEMIA AND LYMPHOMA

(71) Applicant: ASCENTAWITS PHARMACEUTICALS, LTD., Pingshan District, Shenzhen, Guangdong (CN)

(72) Inventors: Yanbin XIE, Shenzhen, Guangdong (CN); Jing HAO, Shenzhen, Guangdong (CN); Tianyang QI, Shenzhen, Guangdong (CN); Fanying MENG, San Francisco, CA (US); Jianxin DUAN, Shenzhen, Guangdong (CN)

(21) Appl. No.: 19/110,239

(22) PCT Filed: Sep. 8, 2023
(86) PCT No.: PCT/CN2023/117606

§ 371 Date: Mar. 10, 2025

(30) Foreign Application Priority Data
Sep. 9, 2022 (CN) 202211105071.9

(51) Int. Cl. A61K 31/675; A61K 31/704; A61K 31/7068; A61P 35/02

(52) U.S. Cl. CPC A61K 31/675; A61K 31/704; A61K 31/7068; A61P 35/02

(57) ABSTRACT

A treatment method, comprising using a drug containing AST-3424, AST-3423 or AST-2870 and a salt thereof, an ester thereof, a solvate thereof, or an isotopic isomer thereof in combination with a drug containing cytarabine or daunorubicin and a salt thereof, an ester thereof, a solvate thereof, or an isotopic isomer thereof to treat leukemia and lymphoma patients. Where the concentration of daunorubicin or cytarabine is far lower than the clinical dosage thereof, there is no need to limit patients to those with higher (overexpression, excess) AKR1C3 protein or RNA expression; the combination of AST-3424 and cytarabine or daunorubicin still has a remarkable inhibitory effect on the in-vitro proliferation of leukemia or lymphoma cells, and the combination has a relatively good inhibitory effect on the in-vitro proliferation of cell lines with different levels of AKR1C3 protein or RNA expression. Therefore, the described combination protocol is expected to be developed into a new treatment protocol for treating leukemia and lymphoma without limiting the level of AKR1C3 enzyme expression in patients.

### TECHNICAL FIELD

The present invention relates to a method for treating tumors, in particular to a combination of AST-3424 with another drug for treating leukemia and lymphoma, and is in the field of tumor therapy.

### BACKGROUND

AST-3424 (CAS No. 2097713-69-2), a prodrug of a DNA alkylating agent that targets the overexpression of aldo-keto reductase family 1 member C3 (AKR1C3) (WO2016145092, WO2017087428), has a structure of the following formula:

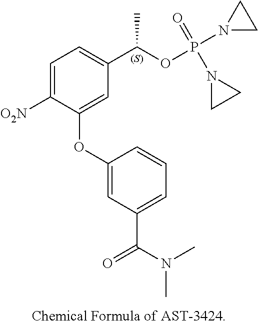

When entering into cancer cells, AST-3424 (also known as OBI-3424 or TH-3424) is activated by AKR1C3 enzyme that is overexpressed in these cancer cells to release a metabolite AST-2660 (also known as AST-2660). AST-3424 itself has low toxicity to cancer cells. In animal models and in vitro pharmacological experiments, its pharmacological effects are associated with AKR1C3 expression levels: the prodrug AST-3424 is metabolized by AKR1C3 enzyme in the presence of NADPH to AST-2660; and its potency is positively correlated with AKR1C3 expression levels (Meng F, Li W F, Jung D, et al. A novel selective AKR1C3-activated prodrug AST-3424/OBI-3424 exhibits broad anti-tumor activity. Am J Cancer Res. 2021; 11(7): 3645-3659; Evans K, Duan J, Pritchard T, et al. OBI-3424, a Novel AKR1C3-Activated Prodrug, Exhibits Potent Efficacy against Preclinical Models of T-ALL. Clin Cancer Res. 2019; 25(14): 4493-4503. doi:10.1158/1078-0432.CCR-19-0551; Wang Y, Liu Y, Zhou C, et al. An AKR1C3-specific prodrug with potent anti-tumor activities against T-ALL. Leuk Lymphoma. 2020; 61(7): 1660-1668. doi:10.1080/10428194.2020.1728746; He P, Wang C, Wang Y, et al. A Novel AKR1C3 Specific Prodrug TH3424 With Potent Antitumor Activity in Liver Cancer [retracted in: Clin Pharmacol Ther. 2021 July; 110(1): 262]. Clin Pharmacol Ther. 2021, 110(1): 229-237. doi:10.1002/cpt.2171).

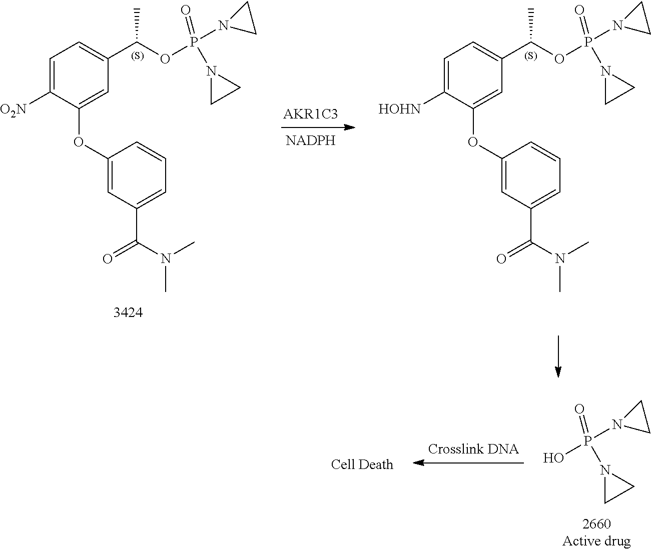

Chemical route of metabolizing AST-3424 to AST-2660 (i.e., 2660 in the figure)

In several clinical trials (NCT03592264 registered in US for castration-resistant prostate cancer (CRPC) and liver cancer; NCT04315324 registered in US for T-Cell Acute Lymphoblastic Leukemia (T-ALL); CTR20191399 registered in China for various solid tumors; and CTR20201915 registered for T-ALL and B-cell acute lymphoblastic leukemia (B-ALL)), the correlation between AKR1C3 expression and the therapeutic efficacy of AST-3424 has been explored. Further, the Phase I study results that are publicly available (Safety, pharmacokinetics, and clinical activity of OBI-3424, an AKR1C3-activated prodrug, in patients with advanced or metastatic solid tumors: A phase 1 dose-escalation study. J Clin Oncol 40, 2022 (suppl 16; abstr 3030), DOI: 10.1200/JCO.2022.40.16_suppl.3030) has shown that, only the patients who have AKR1C3 expression scores (H-scores) as analyzed by the immunohistochemistry (IHC) assay method that exceed a certain value may exhibit a treatment response, and thus, the investigators have determined that the patients with H-score ≥135 will be elected to enroll in the subsequent clinical trials.

Similar conclusions have also been repeated in combination therapy experiments. Particularly, AST-3424 showed excellent efficacy when combined with abiraterone/prednisolone in CDX models with high AKR1C3 expression in VCaP cells, with 5-fluorouracil in CDX models with high AKR1C3 expression in SNU-16 cells, or with sunitinib/gemcitabine in CDX models with high AKR1C3 expression in A498 cells, as compared with its use as monotherapy (Meng F, Li W F, Jung D, et al. A novel selective AKR1C3-activated prodrug AST-3424/OBI-3424 exhibits broad anti-tumor activity. Am J Cancer Res. 2021; 11(7):3645-3659).

Particularly, it has been found in the latest studies that AST-3424 showed excellent inhibitory effects in the hepatocellular carcinoma (HCC) cell lines: HepG2 and PLC/PRF/5 with high AKR1C3 expression, and its combination with oxaliplatin (OXA) or 5-fluorouracil (5-Fu) resulted in significantly enhanced inhibitory effects, suggesting a synergistic effect. By adopting different experimental methods, it was found that AST-3424 monotherapy and combination therapy with OXA or 5-Fu can exert an in-vitro antitumor synergistic effect on liver cancer cells by inhibiting cell proliferation, inducing cell stagnation in the S phase, promoting cell apoptosis induction, and/or inhibiting cell metastasis:

The Western-blot assay demonstrated that the potential mechanism may be related to the upregulation of P21 protein and may not be associated with P-catenin protein expression;

Flow cytometry was used to detect the percentage of cells in different phases. The results suggested that AST-3424 monotherapy and combination therapy with OXA or 5-Fu could block the cells in the S phase. The induction of apoptosis after treatment with AST-3424 monotherapy and combination therapy with OXA or 5-Fu was detected by flow cytometry. The results showed that AST-3424 monotherapy and combination therapy with OXA or 5-Fu may induce apoptosis by upregulating Bax and Caspase3 proteins and downregulating the PARP protein.

The Transwell cell migration and invasion assay demonstrated that AST-3424 monotherapy and combination therapy with OXA or 5-Fu could inhibit the migration and invasion of tumor cells, and the mechanism may be related to the downregulation of MMP-9 proteins.

The effects of AST-3424 monotherapy and combination therapy with OXA or 5-Fu on the TGF-β signaling pathway were investigated. The experimental results suggested that AST-3424 monotherapy and combination therapy with OXA or 5-Fu could reduce the proliferation effect by inhibiting the TGF-β signaling pathway, and the inhibition effect was significant only in the combination of AST-3424 and OXA.

### SUMMARY

Existing studies have shown that the potency of AST-3424 is dependent on the level of AKR1C3 expression, and better efficacy was only observed in patients or animal models with high AKR1C3 expression. Furthermore, the dependency on AKR1C3 expression also has been indicated in the published in vitro cell proliferation experiments and CDX animal models involving the combination therapy with AST-3424 and abiraterone, prednisolone, 5-fluorouracil, sunitinib, or oxaliplatin. These studies have shown that AST-3424 either as monotherapy or in combination therapy requires that the AKR1C3 expression in a patient should reach a predetermined high level (as detected by kits, e.g., by IHC/q-PCR methods).

In combination therapy studies, however, the inventors have found that the inhibitory effects exhibited by the combination of AST-3424 with cytarabine or daunorubicin on the in vitro proliferation of leukemia and lymphoma cells are independent of the AKR1C3 expression levels in the cell lines, and this combination therapy could show excellent inhibitory effects on the in vitro proliferation either with dim or strong AKR1C3 expression levels in the cell lines. To this end, the above-mentioned combination therapy protocol is a new and promising treatment regimen for leukemia and lymphoma, without being limited to the patients with overexpression or high expression of AKR1C3 (which often corresponds to higher RNA quantitative values or higher H-scores on IHC analysis) as disclosed in previous research papers or literature.

This new treatment regimen has significantly lowered the eligibility threshold of the patients who may benefit from AST-3424 therapy. If the patients have a positive expression, they may receive this combination therapy regimen as long as a medical professional has determined after discreet consideration that they may benefit or potentially benefit from the administration with such a therapy regimen.

The present invention provides a method for treating patients with leukemia or lymphoma, comprising administering a medicament selected from AST-3424, AST-3423 or AST-2870 and their salts, esters, solvates or isotope isomers in combination with another medicament selected from cytarabine or daunorubicin and their salts, esters, solvates, or isotope isomers. The expression “AST-3424, AST-3423 or AST-2870 and their salts, esters, solvates or isotope isomers” denotes seven parallel candidates, representing AST-3424, AST-3423, AST-2870, salts of AST-3424, AST-3423 or AST-2870, esters of AST-3424, AST-3423 or AST-2870, solvates of AST-3424, AST-3423 or AST-2870, and isotope isomers of AST-3424, AST-3423 or AST-2870, respectively. The expression “cytarabine or daunorubicin and their salts, esters, solvates, or isotope isomers” denotes six parallel candidates, representing cytarabine, daunorubicin, salts of cytarabine or daunorubicin, esters of cytarabine or daunorubicin, solvates of cytarabine or daunorubicin, and isotope isomers of cytarabine or daunorubicin, respectively.

The present invention also provides use of AST-3424, AST-3423 or AST-2870 and their salts, esters, solvates or isotope isomers in combination with cytarabine or daunorubicin and their salts, esters, solvates, or isotope isomers in the manufacture of a medicament for treating leukemia or lymphoma.

According to the data disclosed in patents WO2016145092 and WO2017087428, AST-3423 is an enantiomer of AST-3424; AST-3424 represents configuration (S); AST-3423 represents configuration (R); and AST-2870 (TH2870) is a racemic mixture of AST-3424 and AST-3423. The three compounds have similar AKRTC3-activated properties and are all catalyzed by AKRTC3 in the presence of NADPH to release a cytotoxic substance AST-2660 that can exert pharmacological efficacy.

Regarding the medicament as described herein, it refers to a medicinal product or formulation. The prepared medicinal product contains a specific dosage range of the active ingredients or their salts or solvates, and/or the prepared medicament is in a particular dosage form or is administered via a particular mode of administration.

The prepared medicinal product, medicament or formulation may further comprise pharmaceutically acceptable auxiliaries or excipients. The medicament may be in any dosage form for clinical administration, such as tablets, suppositories, dispersible tablets, enteric tablets, chewable tablets, orally disintegrating tablets, capsules, sugar-coated preparations, granules, dry powders, oral solutions, small dose injections, lyophilized powder for injection, or infusion solutions. According to the specific dosage form and the mode of administration, the pharmaceutically acceptable auxiliaries or excipients in the medicament may include one or more of diluents, solubilizers, disintegrants, suspending agents, lubricants, binding agents, fillers, flavoring agents, sweeteners, antioxidants, surfactants, preservatives, wrapping agents, and pigments.

Monotherapy refers to single drug therapy. Combination therapy refers to combined drug therapy. Single drug therapy refers to the use of only one anticancer drug in a course of treatment. Combination therapy refers to the simultaneous or sequential use of two or more anticancer drugs in a course of treatment.

Generally speaking, for combination therapy, different administration dosages and administration cycles need to be explored according to the characteristics of the disease and the types of drugs to be used in combination; and only with the exploration according to the above conditions, the combined drug therapy regimen may achieve better therapeutic effect as compared with single drug therapy.

The administration dosages and administration cycles of drugs in both monotherapy and combination therapy regimens need to be explored through clinical trials.

Regarding the compounds as described herein, since their chemical structures contain an organic amine and a P═O double bond, the compounds may also be administered in the form of salts. In other words, the present invention provides pharmaceutically acceptable salts of the compounds. The salts may be basic salts, including the salts of the compounds with an inorganic base (such as alkali metal hydroxide or alkaline earth metal hydroxide) or with an organic base (such as monoethanolamine, diethanolamine or triethanolamine). Alternatively, the salts may be acid salts, including the salts of the compounds formed with an inorganic acid (such as hydrochloric acid, hydrobromic acid, hydroiodic acid, nitric acid, perchloric acid, sulfuric acid or phosphoric acid) or with an organic acid (such as methanesulfonic acid, trifluoromethanesulfonic acid, ethanesulfonic acid, benzenesulfonic acid, p-toluenesulfonic acid, fumaric acid, oxalic acid, maleic acid or citric acid). Analogously, since the compounds may also react with some acids or alcohols to form their esters, they may also be administered in the form of esters. For various reasons, these compounds may also form solvates with some solvents. The solvates are hydrates or alcoholates. Accordingly, the compounds may also be administered in the form of solvates. It is well-known technology in the art to select and prepare acceptable salts, esters, and solvates of a compound.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (¹H), deuterium (²H), tritium (³H), carbon-11 (¹¹C) carbon-12 (¹²C), carbon-13 (¹³C), carbon-14 (¹⁴C), nitrogen-13 (¹³N), nitrogen-14 (¹⁴N), nitrogen-15 (⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I) It will be apprehended that, in a compound as provided herein, any hydrogen can be ²H, namely D, for example, or any carbon can be ¹³C, as example, or any nitrogen can be ¹⁵N, as example, and any oxygen can be ¹⁸O, where feasible in the view of one of ordinary skill in the art. In certain embodiments, an “isotopic variant” of a compound contains unnatural proportions of deuterium (D).

Leukemia, also known as blood cancer, is a malignant tumor in hematopoietic system. It may be caused by abnormal function of hematopoietic tissues in the bone marrow owing to mutations in cellular deoxyribonucleic acid (DNA). There are many types of leukemia, and they are mainly classified based on the types of abnormal blood cells present in the blood. There are various classification methods academically. Typical classification methods include French-American-British (FAB) classification and the new WHO classification scheme promoted by the World Health Organization (WHO). These classification methods can provide guidance on the prognosis and treatment of patients. In clinical practice, leukemia is generally divided into acute leukemia and chronic leukemia, including:
acute lymphoblastic leukemia (abbreviated as “ALL”), which can be diagnosed and divided into different subtypes using immunological techniques based on different differentiation antigens present on the surface of leukemia cells; and it is generally divided into T-cell and B-cell lines, namely: T-Cell acute lymphoblastic leukemia (T-ALL) and B-cell acute lymphoblastic leukemia (B-ALL);
acute myelogenous leukemia (also known as acute myeloid leukemia), a blood cancer in which myeloid white blood cells (rather than lymphoid white blood cells) proliferate abnormally; chronic lymphocytic leukemia (abbreviated as CLL), which mainly includes B-cell chronic lymphocytic leukemia (B-CLL) and T-cell chronic lymphocytic leukemia (T-CLL) that rarely occurs; and
chronic myelogenous leukemia, which is also known as chronic myeloid leukemia (abbreviated as “CML”).

Lymphoma is a malignant tumor originating from lymph nodes or other lymphoid tissues. It can be broadly divided into two types: Hodgkin disease (HD) and non-Hodgkin lymphoma (NHL). Non-Hodgkin lymphoma is broadly divided into two types: B-cell lymphoma and T-cell/natural killer (NK)-cell lymphoma.

B-cell lymphoma is further divided into two major types: precursor B-cell lymphoma and mature B-cell lymphoma. Precursor B-cell lymphoma refers to precursor B-lymphoblastic lymphoma. Clinically-common mature B-cell lymphomas include small cell lymphoma, plasma cell myeloma, extranodal marginal zone B-cell lymphoma, nodal marginal zone B-cell lymphoma, follicular lymphoma, mantle cell lymphoma, diffuse B-cell lymphoma, and Burkitt lymphoma.

Subtypes of T-cell/NK-cell lymphoma also include precursor T-cell lymphoma and mature T-cell lymphoma. Precursor T-cell lymphoma mainly refers to precursor T-lymphoblastic lymphoma. Common mature T-cell lymphomas include T-cell prolymphocytic leukemia, adult T-cell lymphoma, extranodal NK-T lymphoma, anaplastic large cell lymphoma, peripheral T-cell lymphoma, and anaplastic large cell lymphoma.

Particularly, the leukemia includes lymphocytic leukemia (comprising acute lymphoblastic leukemia and chronic lymphoblastic leukemia), and the lymphoma includes lymphoblastic lymphoma.

Further, the leukemia is acute lymphoblastic leukemia, preferably T-cell acute lymphoblastic leukemia, and the lymphoma is T-cell lymphoblastic lymphoma.

Particularly, leukemia includes acute lymphoblastic leukemia, T-cell acute lymphoblastic leukemia (T-ALL), T-cell lymphoblastic leukemia, and acute lymphoblastic leukemia; and lymphoma includes T-cell lymphoblastoma, specifically T-cell lymphoblastic lymphoma.

The daunorubicin or cytarabine is administered in combination therapy at a dose lower than their clinical doses each as a single agent.

Daunorubicin is clinically used in the form of hydrochloride, i.e., daunorubicin hydrochloride. Its dosage form is sterile powder for injection, which is reconstituted in normal saline for injection before use in intravenous injection or infusion. Clinically, it is mainly used for treating:
(1) acute myeloid leukemia: daunorubicin is suitable for treating this type of leukemia at all stages, whether it is used alone or in combination with other anti-tumor drugs; it is also useful for treating promyelocytic leukemia;
(2) acute lymphoblastic leukemia: daunorubicin could achieve a high remission rate in the therapy of this type of leukemia; however, owing to its severe side effects and the availability of other effective treatments, daunorubicin is only suitable for use in patients who have developed resistance to the therapies with other drugs; and
(3) other tumors: it has been observed that daunorubicin has excellent therapeutic effects on neuroblastoma and rhabdomyosarcoma.

Cytarabine is clinically used as cytarabine for injection (cytarabine or cytarabine hydrochloride). Its dosage form is lyophilized powder, which is reconstituted in a benzyl alcohol injection prepared from benzyl alcohol and water (which is generally sold together with lyophilized powder) prior to use in intravenous or subcutaneous injection. It is mainly used for remission induction and maintenance therapy of acute non-lymphoblastic leukemia in adults and children. It also has therapeutic effects on other types of leukemia, such as acute lymphoblastic leukemia and chronic myeloid leukemia (blast crisis phase). It may be used alone or in combination with other anti-tumor drugs to achieve better therapeutic effects.

The blood samples of the patients with leukemia have a positive result in the RNA assay of AKR1C3, and the tumor tissue samples of the patients with lymphoma have a positive result in the AKR1C3 protein assay; or
the patients with leukemia are shown to have a positive result in the RNA assay of AKR1C3, and
the patients with lymphoma are shown to have a positive result in the AKR1C3 protein assay.

Previous studies and experimental papers have demonstrated that AST-3424 monotherapy or combination therapy is limited to animal models or patients with AKR1C3 excess or overexpression. However, the combination of AST-3424 and daunorubicin/cytarabine involved in this treatment regimen/pharmaceutical use for treating leukemia/lymphoma merely requires a positive AKR1C3 protein or RNA assay result, i.e., the patients having AKR1C3 expression. That is, the combination could still lead to significant therapeutic effects even in patients with a positive AKR1C3 protein or RNA assay result.

Excess, i.e., overexpression, means that the expression level of cancer cells is higher than that of normal cells. In this case, it can be denoted as excess or overexpression. Alternatively, when a threshold value is set based on statistic criteria in an artificial way, excess or overexpression means exceeding that threshold value. AKR1C3 excess or overexpression in different tumors (solid tumors or blood cancers) may be found in the following documents or literature: Evans K, Duan J, Pritchard T, et al. OBI-3424, a Novel AKR1C3-Activated Prodrug, Exhibits Potent Efficacy against Preclinical Models of T-ALL. Clin Cancer Res. 2019; 25(14): 4493-4503. doi:10.1158/1078-0432.CCR-19-0551 (which has disclosed AKR1C3 expression levels in acute lymphoblastic leukemia) and Guise C P, Abbattista M R, Singleton R S, et al. The bioreductive prodrug PR-104A is activated under aerobic conditions by human aldo-keto reductase 1C3. Cancer Res. 2010; 70(4):1573-1584. doi: 10.1158/0008-5472. CAN-09-3237 (which has disclosed AKR1C3 expression levels in different solid tumors).

In therapies with the previously-known AKR1C3 enzyme-activated anticancer prodrugs, it is often necessary to measure the AKR1C3 expression level prior to administration, and such treatment regimens have been verified to be effective only for patients with high or excessive AKR1C3 expression. However, the pharmaceutical effects of this new combination therapy provided by this invention are almost independent of the AKR1C3 expression levels. As long as the patients have AKR1C3 expression, i.e., have a positive assay result, they may be effectively treated with such a combination therapy.

Further, the quantitative values of AKR1C3 expression levels as assessed by RNA assay that are expressed as fragments per kilobase million (FPKM) on a log 2 scale (i.e., Log 2 FPKM) in the blood samples of the patients with leukemia are greater than or equal to 0.71; and the quantitative values of AKR1C3 protein in the tumor tissue samples of the patients with lymphoma, which are represented by the ratios of the AKR1C3 protein bands to the reference protein bands in the protein bands as detected by Western blot (WB) assay, are greater than or equal to 0.84;
or
the patients with leukemia are shown to have quantitative values of AKR1C3 expression levels as assessed by RNA assay that are expressed as FPKM on a log 2 scale (i.e., Log 2 FPKM) of greater than or equal to 0.71; and the patients with lymphoma are shown to have quantitative values of AKR1C3 protein that are represented by the ratios of the AKR1C3 protein bands to the reference protein bands in the protein bands as detected by Western blot (WB) assay of greater than or equal to 0.84.

The combination may result in a better effect when the above-mentioned conditions are met.

In other words, the combination therapy of the present invention has broadened the population of patients who may benefit from AST-3424 therapy. Previous prior-art documents (Evans K, Duan J, Pritchard T, et al. OBI-3424, a Novel AKR1C3-Activated Prodrug, Exhibits Potent Efficacy against Preclinical Models of T-ALL. Clin Cancer Res. 2019; 25(14): 4493-4503. doi:10.1158/1078-0432.CCR-19-0551; Guise C P, Abbattista M R, Singleton R S, et al. The bioreductive prodrug PR-104A is activated under aerobic conditions by human aldo-keto reductase 1C3. Cancer Res. 2010; 70 (4): 1573-1584. doi:10.1158/0008-5472.CAN-09-3237; and Safety, pharmacokinetics, and clinical activity of OBI-3424, an AKR1C3-activated prodrug, in patients with advanced or metastatic solid tumors: A phase 1 dose-escalation study. J Clin Oncol 40, 2022 (suppl 16; abstr 3030), DOI: 10.1200/JCO.2022.40.16_suppl.3030) have revealed that the population that may benefit from the treatment of leukemia or solid tumors with AST-3424 is limited to those with excessive expression or overexpression of AKR1C3 protein or RNA (which often corresponds to higher RNA quantitative values or higher H-scores on IHC analysis). However, this new treatment regimen has significantly lowered the eligibility threshold of the patients who may benefit from AST-3424 therapy. The AKR1C3 assay result of the patients before being administered with this combination therapy for treating leukemia/lymphoma does not need to be AKR1C3 overexpression/excess as defined in the previously disclosed documents. This combination therapy may be readily applied when the patients have a positive AKR1C3 assay result or have AKR1C3 expression, as long as a medical professional has determined after discreet consideration that they may benefit or potentially benefit from the administration with such a combination therapy.

The present invention further provides a pharmaceutical combination, which comprises the following substances as active ingredients:
AST-3424, AST-3423 or AST-2870 and their salts, esters, solvates or isotope isomers, in combination with
daunorubicin and their salts, esters, solvates, or isotope isomers, or cytarabine and their salts, esters, solvates, or isotope isomers;
wherein the active ingredients are formulated concomitantly or separately, for use in compatible combination or in simultaneity or separately.

The pharmaceutical combination according to the present invention comprises at least two active ingredients, e.g., AST-3424 and daunorubicin, or AST-3424 and cytarabine. Of course, it may further comprise pharmaceutically acceptable auxiliaries.

Typically, the two active ingredients that characterize the pharmaceutical combination as described herein may be formulated either concomitantly (in single dosage unit) or separately (as a kit).

In general, the two active ingredients that are formulated concomitantly or separately may be administered simultaneously; or depending on their intrinsic properties, they are administered separately at certain time interval that helps to optimize their compatible effect.

The pharmaceutical combination may be a composite medicament, which is a pharmaceutical composition composed of an active ingredient selected from AST-3424, AST-3423, AST-2870 or their salts, esters, solvates, and isotope isomers, and another active ingredient selected from daunorubicin, cytarabine or their salts, esters, solvates, and isotope isomers. Under such circumstances, the active ingredients are formulated concomitantly and administered simultaneously.

The pharmaceutical combination may also be a kit similar to Pfizer's Paxlovid that acts against COVID-19, wherein the active ingredients may be sold together or separately. In this case, the active ingredients are formulated separately. However, the two or more drugs that are formulated separately may be administered simultaneously or separately at certain time interval depending on the intrinsic properties of each ingredient.

As a specific embodiment of the present invention where the active ingredients are formulated separately, one of the formulated medicaments comprises AST-3424, AST-3423, AST-2870 or their salts, esters, solvates, isotope isomers as an active ingredient and pharmaceutically acceptable auxiliaries, and the other medicament comprises daunorubicin, cytarabine or their salts, esters, solvates, isotope isomers as an active ingredient and pharmaceutically acceptable auxiliaries. The pharmaceutical combination composed of the two medicaments may be administered simultaneously or separately at certain time interval depending on the intrinsic properties of each medicament.

In the pharmaceutical combinations, the dose of daunorubicin or cytarabine to be combined is lower than the clinical dose of each single agent.

The present invention further provides a pharmaceutical formulation, which is prepared from the active ingredients of the pharmaceutical combination and pharmaceutically acceptable auxiliaries.

As a particular example of the present invention, the pharmaceutical formulation is in a dosage form selected from granules, tablets, pills, capsules, or injectables.

The present invention further provides use of the pharmaceutical combination and/or the pharmaceutical formulation in the manufacture of an anti-tumor medicament.

As a particular example of the present invention, the tumor is selected from leukemia and lymphoma.

As a preferred example, leukemia includes lymphoblastic leukemia, and lymphoma includes lymphoblastic lymphoma; preferably, the leukemia is acute lymphoblastic leukemia, more preferably T-cell acute lymphoblastic leukemia (T-ALL), and the lymphoma is T-cell lymphoblastic lymphoma.

### BRIEF DESCRIPTION OF THE DRAWINGS

FIG. 1 is a curve showing the inhibitory effects of the three drugs on cell proliferation in five cancer cell lines.

FIG. 2 shows the experimental results regarding the effects of AST-3424 and daunorubicin or cytarabine each as monotherapy or in combination therapy on the proliferation of SUP-T1 cells.

FIG. 3 shows the experimental results regarding the effects of AST-3424 and daunorubicin or cytarabine each as monotherapy or in combination therapy on the proliferation of CCRF-CEM cells.

FIG. 4 shows the experimental results regarding the effects of AST-3424 and daunorubicin or cytarabine each as monotherapy or in combination therapy on the proliferation of PF-382 cells.

FIG. 5 shows the experimental results regarding the effects of AST-3424 and daunorubicin or cytarabine each as monotherapy or in combination therapy on the proliferation of Jurkat, Clone E6-1 cells.

FIG. 6 shows the experimental results regarding the effects of AST-3424 and daunorubicin or cytarabine each as monotherapy or in combination therapy on the proliferation of MOLT-4 cells.

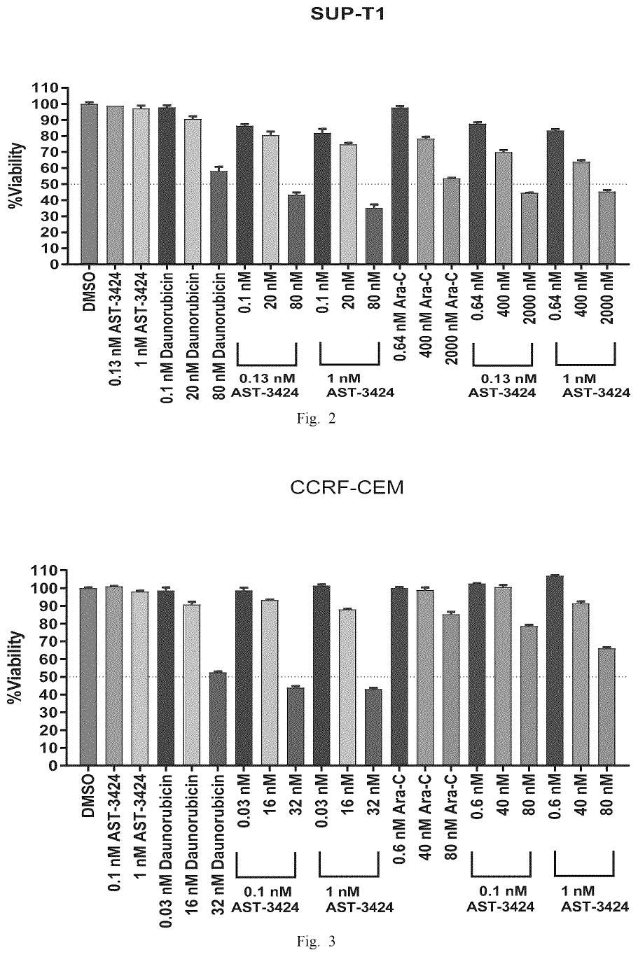

In FIG. 2, Ara-c represents cytarabine, Daunorubicin represents daunorubicin, and the 4th, 5th, and 6th column charts from left to right represent the cell viability percentage values after the monotherapy with 0.1 nM, 20 nM, and 80 nM daunorubicin, respectively; the 7th, 8th, and 9th column charts on the right side correspond to the cell viability percentage values after the combination therapy of 0.13 nM AST-3424 with 0.1 nM, 20 nM, and 80 nM daunorubicin, respectively; and the 10th, 11th, and 12th column charts on the right side correspond to the cell viability percentage values after the combination therapy of 1 nM AST-3424 with 0.1 nM, 20 nM, and 80 nM daunorubicin, respectively. Similar meanings may also apply to FIGS. 2 to 6.

### EXAMPLES

### 1. In Vitro Cell Experiments

### 1.1 In Vitro Cell Proliferation Assays Performed to Study the Inhibition of Monotherapy Procedure

On day 1, cells were seeded in 96-well cell culture plates (at 180 μL/well).

On day 2, 20 μL of a 10-fold test concentration of the working solution of the compound (AST-3424) was added. The cell plates were gently agitated, and were then placed in an incubator at 37 °C for further culture.

On day 3, 20 μL of a 10-fold test concentration of the solution of the compound (cytarabine and daunorubicin) was added. The cell plates were gently agitated, and were then placed in an incubator at 37 °C for further 72 hours.

After incubation, to the cell plates was added a reagent prepared according to the Protocol for a 35 CellTiter-Glo Reagent (50 μL/well). The plates were thoroughly agitated and were then incubated at room temperature in the dark for 10 minutes.

The cell plates were placed in a plate reader for analysis to detect chemiluminescence and record the data.

### Data Processing

The readings in each well should be converted into cell viability, which was calculated by the following formula:

$$Cell viability ( \% ) = \frac{Sample cell reading - blank control}{Solvent control cell - blank control} \times 100 \%$$

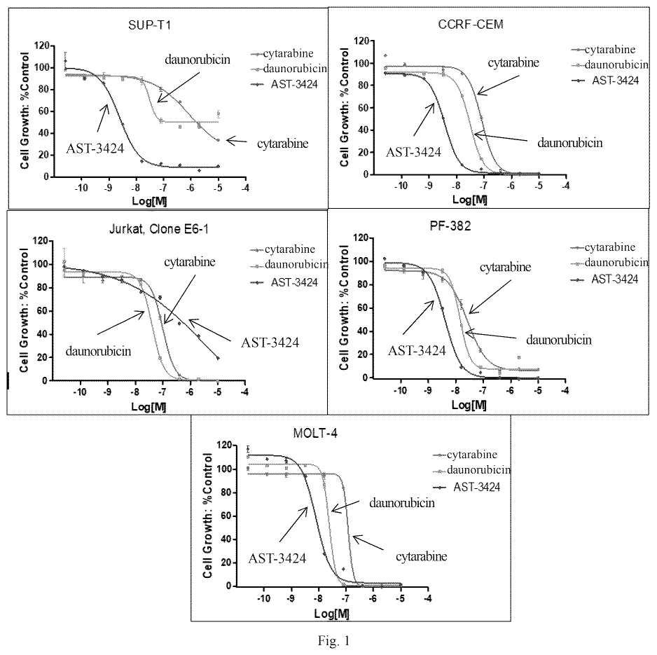

The processed data was analyzed with nonlinear regression using a GraphPad Prism 5 analysis software to obtain the dose-response curve (as shown in FIG. 1), and to calculate the half-maximal lethal concentrations (IC₅₀) of the compounds, as shown in Table 1 below.

TABLE 1
Data on the inhibitory effects of different agents
on cell proliferation in different cell lines

| | IC₅₀ of Different Compounds, in nmol/L | | |
|---|---|---|---|
| | | Daunorubicin | |
| Cell Line | Cytarabine | hydrochloride | AST-3424 |
| SUP-T1 | 856 | 30.6 | 2.47 |
| CCRF-CEM | 82.2 | 30.7 | 3.68 |
| Jurkat, Clone E6-1 | 100 | 41.0 | 4620 |
| PF-382 | 26.4 | 14.2 | 4.09 |
| MOLT-4 | 120 | 24.2 | 8.04 |

Cytarabine, daunorubicin and the compound AST-3424 all showed good inhibitory effects in the five cell lines including CCRF-CEM, PF-382, Jurkat, Clone E6-1, SUP-T1, and MOLT-4. AST-3424 exhibited an in vitro cytotoxicity superior to that of daunorubicin and cytarabine in the four other cell lines except Jurkat, Clone E6-1.

### 1.2 In Vitro Cell Proliferation Assays Performed to Study the Inhibition of Combination Therapy

### Procedure

On day 1, cells were seeded in 96-well cell culture plates (at 180 μL/well).

On day 2, 10 μL of a 20-fold test concentration of the AST-3424 solution (20-fold concentration of either of the two concentrations IC₂₅ and IC₁₀) was added. The cell plates were gently agitated, and were then placed in an incubator at 37 °C for further culture.

On day 3, to the combination therapy wells was added 10 μL of a 20-fold test concentration of the compound (cytarabine or daunorubicin) (20-fold concentration of each of the three concentrations IC₅₀, IC₂₅, and ICs), and to the monotherapy wells was added 10 μL of a culture medium containing 2% DMSO. The cell plates were gently agitated, and were then placed in an incubator at 37 °C for further 72 h.

After incubation, a reagent prepared according to the instructions for the CellTiter-Glo Reagent was added (at 50 μL/well). The cell plates were agitated thoroughly and were then incubated at room temperature in the dark for 10 minutes.

The cell plate was placed in a plate reader for analysis to detect chemiluminescence and record the data.

### Data Processing

The readings in each well should be converted into cell viability, which was calculated by the formula as described in section 1.1.

The final viability percentages under different conditions were plotted into column charts. FIGS. 2-6 show the effects of AST-3424, daunorubicin and cytarabine, in monotherapy and in combination therapy, on the proliferation of the five cell lines including SUP-T1, CCRF-CEM, PF-382, Jurkat, Clone E6-1, and MOLT-4.

### Assay Conclusion

As can be seen from the analysis of FIG. 2, which shows the effects of AST-3424, daunorubicin and cytarabine, in monotherapy and in combination therapy, on the proliferation of SUP-T1 cells:
1. 0.13 nM (nM means a concentration in nmol/L, the same below) and 1 nM AST-3424 in monotherapy showed no effect on the proliferation of SUP-T1 cells;
2. Three different concentrations of daunorubicin in combination therapy with either 0.13 nM or 1 nM AST-3424 resulted in an AST-3424 dose-dependent decrease in cell viability. 80 nM daunorubicin in combination with the two concentrations of AST-3424 showed higher potency than 0.1 nM and 20 nM daunorubicin.
3. Three different concentrations of cytarabine in combination therapy with either 0.13 nM or 1 nM AST-3424 resulted in a decrease in cell viability. 0.64 nM and 400 nM cytarabine in combination therapy respectively with AST-3424 showed an AST-3424 dose-dependent decrease.

As can be seen from the analysis of FIG. 3, which shows the effects of AST-3424, daunorubicin and cytarabine, in monotherapy and in combination therapy, on the proliferation of CCRF-CEM cells:
1. 0.1 nM and 1 nM AST-3424 in monotherapy showed no effect on the proliferation of CCRF-CEM cells;
2. As compared with the 32 nM daunorubicin monotherapy group, the combination therapy thereof with 0.1 nM or 1 nM AST-3424 resulted in a decrease in cell viability, with no AST-3424 dose-dependency. 32 nM daunorubicin in combination with the two concentrations of AST-3424 showed higher potency than 16 nM daunorubicin. The combination therapy with 0.03 nM daunorubicin showed no effect.
3. As compared with the 80 nM cytarabine monotherapy, the combination therapy thereof with either 0.1 nM or 1 nM AST-3424 resulted in an AST-3424 dose-dependent decrease in cell viability. 80 nM cytarabine in combination with the two concentrations of AST-3424 showed higher potency than 40 nM cytarabine. The combination therapy with 0.6 nM cytarabine showed no effect.

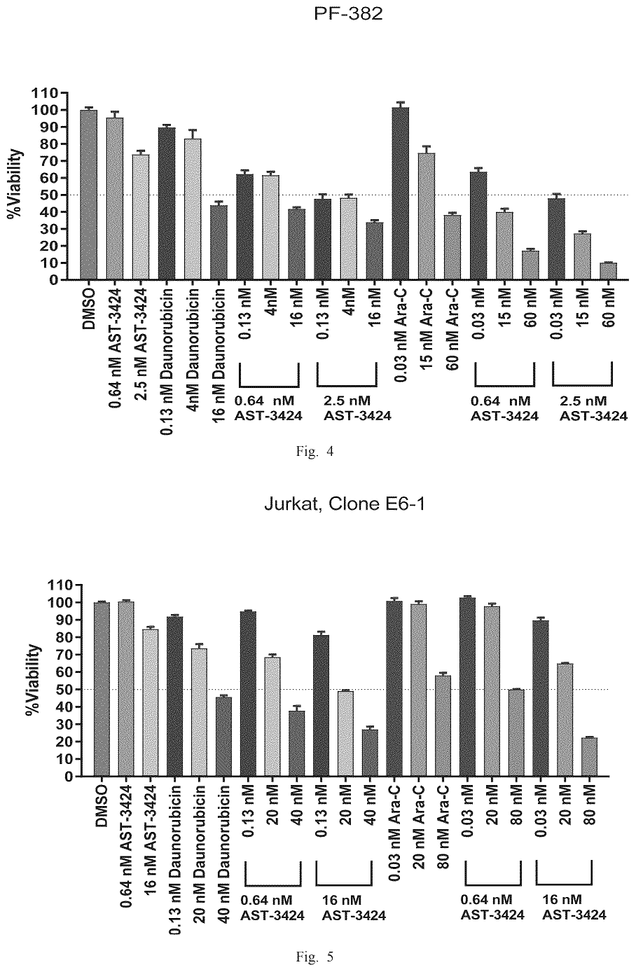

As can be seen from the analysis of FIG. 4, which shows the effects of AST-3424, daunorubicin and cytarabine, in monotherapy and in combination therapy, on the proliferation of PF-382 cells:
1. 0.64 and 2.5 nMAST-3424 in monotherapy showed no significant effect on the proliferation of PF-382 cells;
2. Three different concentrations of daunorubicin in combination therapy with either 0.64 nM or 2.5 nM AST-3424 resulted in an AST-3424 dose-dependent decrease in cell viability;
3. Three different concentrations of cytarabine in combination therapy with either 0.64 nM or 2.5 nM AST-3424 resulted in an AST-3424 dose-dependent decrease in cell viability.

As can be seen from the analysis of FIG. 5, which shows the effects of AST-3424, daunorubicin and cytarabine, in monotherapy and in combination therapy, on the proliferation of Jurkat, CloneE6-1 cells:
1. 0.64 nM and 16 nM AST-3424 in monotherapy showed no significant effect on the proliferation of Jurkat, CloneE6-1 cells;
2. Three different concentrations of daunorubicin in combination therapy with either 0.64 nM or 16 nM AST-3424 resulted in an AST-3424 dose-dependent decrease in cell viability;
3. Three different concentrations of cytarabine in combination therapy with either 0.64 nM or 16 nM AST-3424 resulted in an AST-3424 dose-dependent decrease in cell viability.

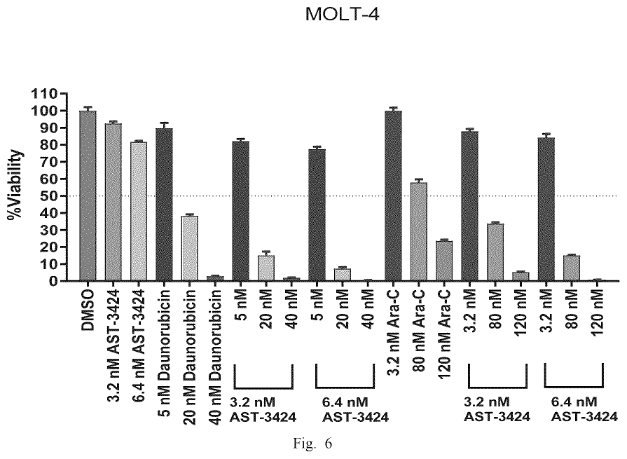

As can be seen from the analysis of FIG. 6, which shows the effects of AST-3424, daunorubicin and cytarabine, in monotherapy and in combination therapy, on the proliferation of MOLT-4 cells:
1. 3.2 nM and 6.4 nM AST-3424 in monotherapy showed no significant effect on the proliferation of MOLT-4 cells;
2. 40 nM daunorubicin showed very significant potency on cell proliferation, and the effect of the combination therapy on cell viability cannot be determined;
3. As compared with the 5 nM or 20 nM daunorubicin monotherapy group, the combination therapy thereof with either 3.2 nM or 6.4 nM AST-3424 resulted in an AST-3424 dose-dependent decrease in cell viability. 20 nM daunorubicin in combination with the two concentrations of AST-3424 showed higher potency than 5 nM daunorubicin;
4. Three different concentrations of cytarabine in combination therapy with 3.2 nM or 6.4 nM AST-3424 resulted in an AST-3424 dose-dependent decrease in cell viability. 80 nM and 120 nM cytarabine in combination with the two concentrations of AST-3424 showed higher potency than 3.2 nM cytarabine.

### II. Information on the Cell Lines and Drugs

### 2.1 Information on the Cell Lines

1. MOLT-4 and MOLT-3 were derived from a 19-year-old male patient with acute lymphoblastic leukemia in relapse who had previously received polydrug combination chemotherapy. The MOLT-4 cell line was originated from T lymphocytes, wherein there is a G→A mutation in Codon 248 of the p53 gene with no expression of p53, immunoglobulin or Epstein-Barr virus, resulting in high levels of terminal deoxyribosyl transferase, and expression of CD1 (49%), CD2 (35%), CD3 A (26%) B (33%) C (34%), CD4 (55%), CD5 (72%), CD6 (22%), and CD7 (77%). The cell is a human acute lymphoblastic leukemia cell, and the corresponding disease is human T-cell acute lymphoblastic leukemia, or T-ALL.
2. PF-382 is a human T-cell acute lymphoblastic leukemia cell, and the corresponding disease is human T-cell acute lymphoblastic leukemia, or T-ALL.
3. Jurkat, Clone E6-1 is a clone of the Jurkat-FHCRC cell line, a derivative of the Jurkat cell line, which was established from the peripheral blood of a 14-year-old male patient suffering from T-cell acute leukemia. It is a human T lymphocytic leukemia cell, and the corresponding disease is T-ALL.
4. SUP-T1 is a human T lymphoblastoma cell, and the corresponding disease is T-cell Lymphoblastic Lymphoma (T-LBL).
5. CCRF-CEM is a human acute lymphoblastic leukemia, T lymphocyte serum culture cell, which was obtained in November 1964 from the peripheral blood leukocytes of a 4-year-old white female patient suffering from acute lymphoblastic leukemia. The cell is a human T-cell acute lymphoblastic leukemia cell, and the corresponding disease is T-cell acute lymphoblastic leukemia, or T-ALL.

### 2.2 AKR1C3 Expression Level Data of the Cells

The AKR1C3 enzyme and RNA expression levels of the above five leukemia and lymphoma cells were tested using the method reported in the literature. The results are shown in Table 2 below.

TABLE 2
AKR1C3 protein and RNA expression data in different cell lines

| | RNA | Protein | IC₅₀ | IC₅₀ |
|---|---|---|---|---|
| Cell line | Expression | Expression | (nmol/L)¹ | (nmol/L)² |
| SUP-T1 | 5.96 | 6.08 | 2.47 | 3.7 |
| CCRF-CEM | 4.10 | 3.95 | 3.68 | 14.9 |
| Jurkat, | 0.71 | 0.84 | 4620 | 24.0 |
| Clone E6-1 | | | | |
| PF-382 | 5.16 | 4.9 | 4.09 | 3.0 |
| MOLT-4 | 1.91 | 4.19 | 8.04 | 10.0 |

AKR1C3 RNA expression values were obtained according to the method as described in the literature (Meng, F, Li, W. F., Jung, D., Wang, C. C., Qi, T., Shia, C. S., Hsu, R. Y., Hsieh, Y. C., & Duan, J. (2021). A novel selective AKR1C3-activated prodrug AST-3424/OBI-3424 exhibits broad anti-tumor activity. American journal of cancer research, 11(7),3645-3659). RNA Seq was used to analyze the AKR1C3 RNA expression level, and quantification was performed by using Log 2 FPKM. The data in the table are the values quantified by Log 2 FPKM.

The AKR1C3 protein expression value is the ratio of the AKR1C3 protein bands to the reference protein bands in the protein bands validated by Western blot (WB) assay. In this example, the protein bands of AKR1C3 and α-Tubulin were processed with an Image J software to measure grayscale values, and then to calculate the ratio of AKR1C3/α-Tubulin as the quantitative value of the relative protein expression. Comparison was made among different cell lines.

The data denoted with superscript 1 represent the assay results of AST-3424 obtained in the experiments of the present invention, see section 1.1 In vitro cell proliferation assays performed to study the inhibition of monotherapy.

The data denoted with superscript 2 represent the assay results of AST-3424 obtained in the experiments that were conducted previously.

### 2.3 Information on the Drugs

Both daunorubicin and cytarabine are drugs used in clinical practice to treat leukemia or other tumors. After investigation, we have compared and converted their doses, and the results are shown in Table 3 below. It was found that the dose of the combination regimen is far lower than the clinical dose of each single agent.

TABLE 3
Clinical dose data of daunorubicin and cytarabine

| | | Actually- | Plasma Cmax/ | Molar | Actually- |
|---|---|---|---|---|---|
| | Typical | administered dose | Steady | concentration | applied molar |
| | clinical | found after | concentration | (μM) after | concentration |
| Compound | dose | consultation | (μg/mL) | conversion | (μM) |
| daunorubicin | 30-60 mg/m², | 44 mg/m², | 26 μg/mL | 49 | ≤0.08 |
| | administration | administration on | (Cmax) | | |
| | for 3 | day 1, 3, 5 | | | |
| | successive | | | | |
| | days | | | | |
| cytarabine | 3 g/m², | 1.8-3 g/m², | 8-24 μg/mL | 32-97 | ≤2 |
| | q12 h, | administration | (steady | | |
| | administration | with 12 times | concentration) | | |
| | for 2-6 | | | | |
| | days | | | | |

### SUMMARY

The combination of AST-3424 at different concentrations either with daunorubicin or cytarabine resulted in an AST-3424 dose-dependent decrease in the viability of the cancer cells used in the assays.

The combination effects on MOLT-4, PF-382 and Jurkat Clone E6-1 cells were better than those on SUP-T1 and CCRF-CEM cells.

In specific leukemia and lymphoma cell lines, the cell lines were detected to have different AKR1C3 expression levels: the quantitative values of the AKR1C3 protein and RNA expression were in the ranges of 0.84-6.08 and 0.71-5.96, respectively. Within such broad ranges of expression levels, the combination of AST-3424 with daunorubicin or cytarabine showed a strong in vitro efficacy that is independent of the AKR1C3 expression level.

The test concentrations of daunorubicin and cytarabine used in the in vitro combination therapy assays were much lower than their clinical doses.

In conclusion, the assays have revealed that the new treatment regimen for leukemia/lymphoma patients does not necessarily require patients to have high (overexpressed, excessive) AKR1C3 protein or RNA expression. A positive AKR1C3 protein or RNA assay result may suffice to suggest that the patients would benefit from this treatment regimen. Thus, the new treatment regimen has significantly lowered the eligibility threshold for the patients who may potentially benefit from the administration with such a combination therapy. The combination regimen of this invention may be used once the patients have a positive test result suggesting that they have AKR1C3 expression, on condition that a medical professional has identified that the patients would benefit or potentially benefit from the treatment with this combination regimen.

1. A method for treating patients with leukemia or lymphoma, comprising administering a medicament selected from AST-3424, AST-3423 or AST-2870 and their salts, esters, solvates or isotope isomers, in combination with another medicament selected from cytarabine or daunorubicin and their salts, esters, solvates or isotope isomers.

2. The method according to claim 1, wherein the leukemia comprises lymphoblastic leukemia and the lymphoma comprises lymphoblastic lymphoma; preferably, the leukemia is acute lymphoblastic leukemia, and more preferably T-cell acute lymphoblastic leukemia (T-ALL), and the lymphoma is T-cell lymphoblastic lymphoma.

3. The method according to claim 1, wherein the daunorubicin or cytarabine is administered in combination therapy at a dose lower than the clinical dose of each single agent.

4. The method according to claim 1, characterized in that:
the blood samples of the patients with leukemia have a positive result in the RNA assay of AKR1C3, and the tumor tissue samples of the patients with lymphoma have a positive result in the AKR1C3 protein assay; or
the patients with leukemia are shown to have a positive result in the RNA assay of AKR1C3, and
the patients with lymphoma are shown to have a positive result in the AKR1C3 protein assay.

5. The method according to claim 4, characterized in that:
the quantitative values of AKR1C3 expression levels as assessed by RNA assay that are expressed as fragments per kilobase million (FPKM) on a log₂ scale in the blood samples of the patients with leukemia are greater than or equal to 0.71;
the quantitative values of AKR1C3 protein in the tumor tissue samples of the patients with lymphoma, which are represented by the ratios of the AKR1C3 protein bands to the reference protein bands in the protein bands as detected by Western blot (WB) assay, are greater than or equal to 0.84;
or
the patients with leukemia are shown to have quantitative values of AKR1C3 expression levels as assessed by RNA assay that are expressed as FPKM on a log₂ scale of greater than or equal to 0.71;
the patients with lymphoma are shown to have quantitative values of AKR1C3 protein that are represented by the ratios of the AKR1C3 protein bands to the reference protein bands in the protein bands as detected by Western blot (WB) assay of greater than or equal to 0.84.

6-10. (canceled)

11. A pharmaceutical combination, characterized in comprising, as active ingredients:
AST-3424, AST-3423 or AST-2870 and their salts, esters, solvates or isotope isomers, in combination with
daunorubicin and its salts, esters, solvates, or isotope isomers, or
cytarabine and their salts, esters, solvates, or isotope isomers
wherein, the active ingredients are formulated concomitantly or separately for use in compatible combination or in simultaneity or separately.

12. The pharmaceutical combination according to claim 11, wherein the dose of daunorubicin and cytarabine used in the combination is lower than the clinical dose of each single agent.

13. A pharmaceutical formulation, characterized in that it is prepared from the active ingredients of the pharmaceutical combination according to claim 11 and pharmaceutically acceptable auxiliaries.

14. The pharmaceutical formulation according to claim 13, wherein the pharmaceutical formulation is in a dosage form selected from granules, tablets, pills, capsules or injectables.

15. A method for treating tumor, comprising administering the pharmaceutical combination according to claim 11.

16. The method according to claim 15, wherein the tumor is selected from leukemia and lymphoma.

17. The method according to claim 16, wherein the leukemia comprises lymphoblastic leukemia, and the lymphoma comprises lymphoblastic lymphoma; preferably, the leukemia is acute lymphoblastic leukemia, and more preferably T-cell acute lymphoblastic leukemia (T-ALL), and the lymphoma is T-cell lymphoblastic lymphoma.
